(19) United States
(12) Patent Application Publication
(10) Pub. No.: US 2024/0311657 A1
(43) Pub. Date: Sep. 19, 2024

(54) APPARATUS AND METHOD FOR PREDICTING ANTIMICROBIAL PEPTIDE FUNCTION USING ARTIFICIAL INTELLIGENCE

(71) Applicant: GWANGJU INSTITUTE OF SCIENCE AND TECHNOLOGY, Gwangju (KR)

(72) Inventors: Hojung NAM, Gwangju (KR); Hansol LEE, Gwangju (KR)

(21) Appl. No.: 18/424,776

(22) Filed: Jan. 27, 2024

(30) Foreign Application Priority Data
Mar. 16, 2023 (KR) 10-2023-0034820

(51) Int. Cl. G06N 5/022; G16B 15/30; G16B 40/20

(52) U.S. Cl. CPC G06N 5/022; G16B 15/30; G16B 40/20

(57) ABSTRACT

Provided are an apparatus and method for determining antimicrobial peptide function using artificial intelligence. The artificial intelligence, which is a bidirectional encoder representation from transformer (BERT)-based model that is pre-trained through unsupervised learning using large amounts of protein sequences may be additionally trained (fine-tuned) using labeled antimicrobial peptide and non-antimicrobial peptide sequences to improve accuracy of determining the peptide function.

### CROSS-REFERENCE TO RELATED APPLICATIONS

The present application claims priority under 35 U.S.C. 119 and 35 U.S.C. 365 to Korean Patent Application No. 10-2023-0034820 (filed on 16 Mar. 2023), which is hereby incorporated by reference in its entirety.

### BACKGROUND

The present disclosure relates to prediction of antimicrobial peptide (AMP) function, and more particularly, to an AMP function prediction technology using artificial intelligence.

Antibiotics are substances used to prevent bacterial infection or treat bacterial diseases. The antibiotics are used to suppress germ (bacteria) by killing the or hindering growth of bacteria.

However, since the antibiotics act specifically only on bacteria, there is a limitation in that the antibiotics kill not only pathogenic bacteria but also beneficial bacteria, and various side effects such as development of antibiotic resistance in bacteria having DNA mutations in which the effects of the antibiotics are not effective.

To respond to these antibiotic-resistant bacteria, antimicrobial peptides (AMPs) have recently received attention, and related researches are being actively conducted.

The antimicrobial peptides are short proteins that occur naturally in all areas of life.

FIG. 5 is a schematic view illustrating an effect of an antimicrobial peptide.

An antimicrobial peptide (11) destroys (12) cell membranes through electrostatic interactions with common components of microorganisms such as cell membranes or inhibit (13) interactions within cells to cause cell lysis.

A non-specific mechanism of the antimicrobial peptide, which is different from that of the antibiotics, is emerging as a new and viable alternative for treatment of infections caused by bacteria, viruses, and fungi and for treatment of cancer.

Thus, it is essential to find candidate antimicrobial peptides and their properties that are capable of responding to antibiotic-resistant bacteria.

Recently, in-silico methods have been attempted to predict drug activity based on simulation of a computer rather than actual living organisms or cells.

In particular, as artificial intelligence learning methods such as deep learning and machine learning are developed, studies are being conducted to determine activation of the antimicrobial peptide in advance by learning the antimicrobial peptide to the artificial intelligence.

However, such studies using the artificial intelligence according to the related art have faced limitations with identifying function and sequence characteristics of the antimicrobial peptide.

The inventors of the present disclosure have been tried to make efforts for solving limitations of predicting an antimicrobial peptide function using the artificial intelligence in the related art. After much effort, the present disclosure has been completed to complete an apparatus and method for predicting the antimicrobial peptide function, which are capable of not only predicting the antimicrobial peptide function but also identifying which amino acids of the antimicrobial peptide are important in its function.

### SUMMARY

Embodiments provide an apparatus and method for predicting whether a peptide sequence has antimicrobial peptide function or not using artificial intelligence.

Embodiments also provide an effect of reducing time and cost that are required to screen antimicrobial agents directly in a biological/chemical laboratory by using computer-based artificial intelligence.

Other objects that are not specified in the present invention will be additionally considered within a range that is easily deduced from the following detailed description and its effects.

In one embodiment, an apparatus for predicting antimicrobial peptide function using artificial intelligence including a controller includes: a tokenization unit configured to tokenize an input amino acid sequence for each amino acid and add a class token representing overall characteristics of the amino acid sequence to the tokenized amino acid sequence; an artificial intelligence unit configured to perform a vector operation on the tokenized amino acid sequence for each token so as to generate a final sequence embedding vector by being pre-trained by a natural language processing (NLP) model by inputting unlabeled protein sequences and additionally learned by labeled antimicrobial peptides (AMP) and non-antimicrobial peptides (non-AMP); and an output unit configured to determine the antimicrobial peptide function of the input amino acid sequence by performing a fully connected (FC) layer operation on the sequence embedding vector for the class token of the final sequence embedding vector.

The artificial intelligence unit may include a bidirectional encoder representation from transformer (BERT) model.

The apparatus may further include a position information encoding unit configured to encode position information of each token in the sequence tokenized by the tokenization unit.

The artificial intelligence unit may be configured to calculate how related each token of the tokenized sequence is to other tokens by a self-attention mechanism.

The artificial intelligence unit may be configured to generate the final sequence embedding vector by a multi-head attention mechanism that applies a weighted average of a plurality of attention vectors to each token of the tokenized sequence.

In another embodiment, a method for predicting antimicrobial peptide function using artificial intelligence includes: preparing the artificial intelligence of the controller, which is a pre-trained natural language processing model, using large-scale unlabeled protein sequences as input;
additionally learning the artificial intelligence by labeled antimicrobial peptide (AMP) sequences and non-antimicrobial peptide (non-AMP) sequences; tokenizing the input amino acid sequence for each amino acid and adding a class token that represents overall characteristics of the amino acid sequence to the tokenized amino acid sequence to input the class token; generating a final sequence embedding vector by performing a vector operation on the tokenized amino acid sequence, to which the class token is added for each token, by the artificial intelligence; and determining the antimicrobial peptide function of the amino acid sequence by performing a fully connected (FC) layer operation on the sequence embedding vector for the class token of the final sequence embedding vector.

The artificial intelligence of the controller may include a bidirectional encoder representation from transformer (BERT) model.

The method may further include, after the inputting of the class token, encoding the positional information of each token in the tokenized amino acid sequence.

The artificial intelligence of the controller may calculate how related each token of the tokenized sequence is to other tokens by a self-attention mechanism.

The artificial intelligence of the controller may generate the final sequence embedding vector by a multi-head attention mechanism that applies a weighted average of a plurality of attention vectors to each token of the tokenized sequence.

The details of one or more embodiments are set forth in the accompanying drawings and the description below. Other features will be apparent from the description and drawings, and from the claims.

### BRIEF DESCRIPTION OF THE DRAWINGS

FIG. 1 is a schematic structural view illustrating an apparatus for predicting antimicrobial peptide function using artificial intelligence according to an embodiment.

FIG. 2 is a more detailed structural view illustrating the apparatus for predicting the antimicrobial peptide function using the artificial intelligence according to an embodiment.

FIGS. 3A, 3B, 3C and 3D illustrate an example of attention value analysis results according to an embodiment.

FIG. 4 is a schematic flowchart illustrating a method for predicting antimicrobial peptide function using artificial intelligence according to another embodiment.

FIG. 5 is a view illustrating an example of an action of antimicrobial peptide.

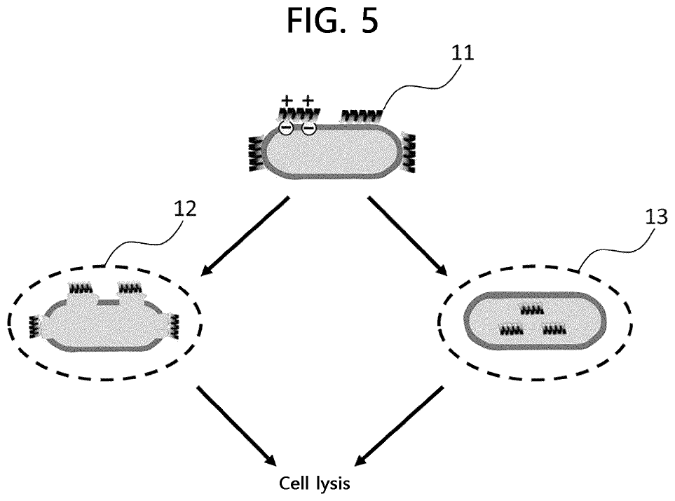

※ The attached drawings are presented for purposes of explanation only, and the technical scope of the present invention is not limited thereto.

### DETAILED DESCRIPTION OF THE EMBODIMENTS

Hereinafter, a configuration of the present invention according to various embodiments of the present invention and effects resulting from the configuration will be described with reference to the drawings. Moreover, detailed descriptions related to well-known functions or configurations will be omitted in order to avoid obscuring subject matters of the present invention.

It will be understood that although the terms such as ‘first’ and ‘second’ are used herein to describe various elements, these elements should not be limited by these terms. These terms are used only to distinguish one component from other components. For example, a first element referred to as a first element in an embodiment can be referred to as a second element in another embodiment without departing from the scope of the appended claims. The terms of a singular form may include plural forms unless referred to the contrary. Unless terms used in embodiments of the present invention are differently defined, the terms may be construed as meanings that are commonly known to a person skilled in the art.

Hereinafter, a configuration of the present invention according to various embodiments of the present invention and effects resulting from the configuration will be described with reference to the drawings.

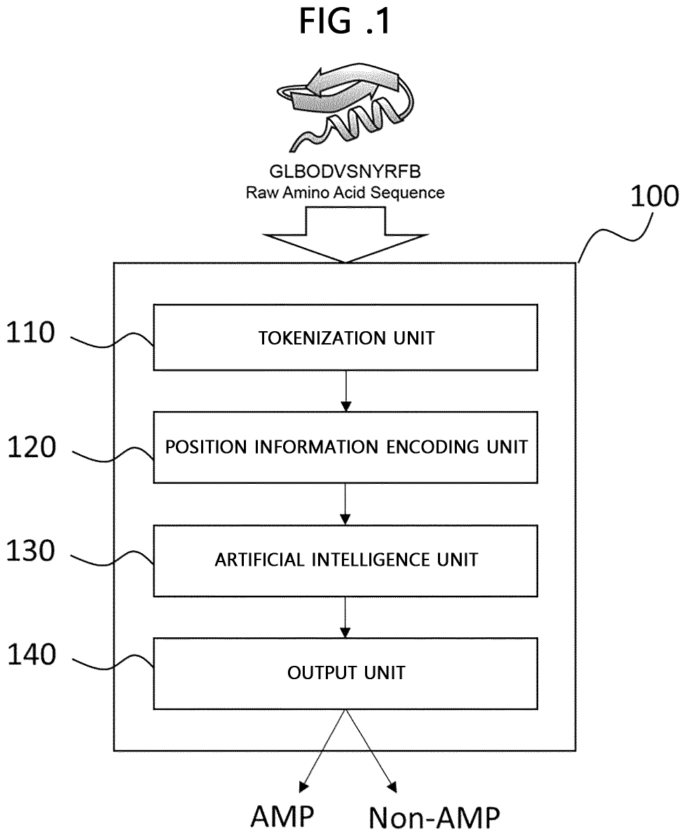

FIG. 1 is a schematic structural view illustrating an apparatus for predicting antimicrobial peptide function using artificial intelligence according to an embodiment.

An apparatus 100 for predicting antimicrobial peptide function according to an embodiment may include a controller (not shown) including one or more processors and a memory. The controller may include a tokenization unit 110, a position information encoding unit 120, an artificial intelligence unit 130, and an output unit 140.

Each of the processors in the controller may be provided as artificial intelligence to operate, and the memory may store program codes and necessary data to run the processor.

The tokenization unit 110 may tokenize amino acid sequence of an analysis target. The position information encoding unit 120 may include position information for each token of the tokenized amino acid sequence.

The artificial intelligence unit 130 may generate a final sequence embedding by performing a vector operation on the tokenized amino acid sequence. The output unit 140 may output whether the corresponding amino acid sequence has antimicrobial peptide function by performing fully connected (FC) layer calculation on the sequence embedding.

For this, the artificial intelligence unit 130 is pre-trained with large-scale protein sequences and fine-tuned with AMP and non-AMP sequences.

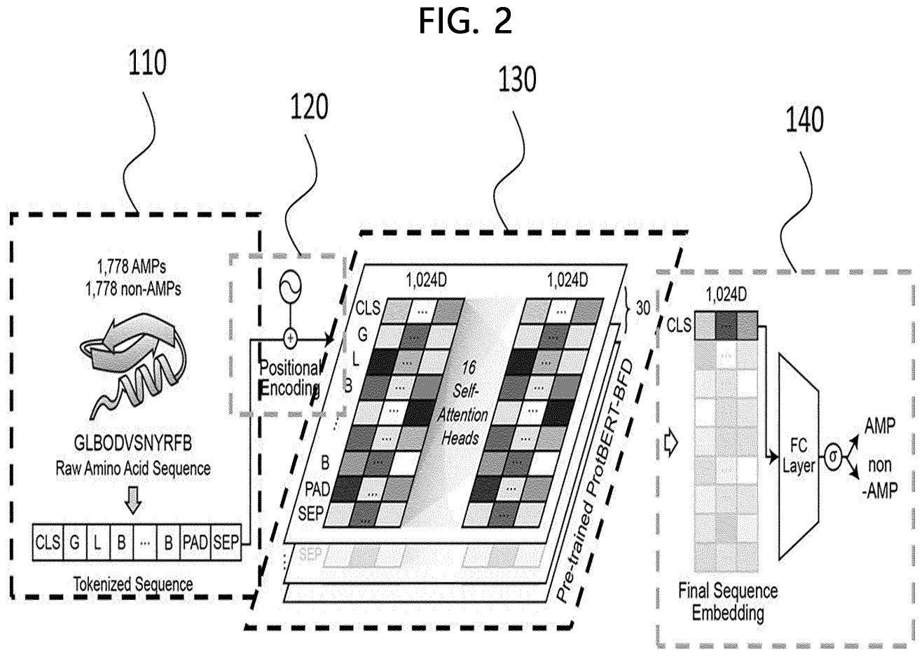

FIG. 2 is a more detailed structural view illustrating the apparatus for predicting the antimicrobial peptide function using the artificial intelligence according to an embodiment.

The tokenization unit 110 may tokenize the amino acid or peptide sequence of the analysis target.

The artificial intelligence unit 130 may be configured using an NLP model. Thus, just as the artificial intelligence unit tokenizes sentences for natural language processing, the amino acid sequence may be tokenized by the tokenization unit 110.

The tokenization unit 110 according to an embodiment may use word-based tokenization as a tokenization method. That is, when the protein sequence is a sentence, each amino acid in the amino acid sequence may be treated as a word.

In addition to the tokenization of the amino acid sequence, the tokenization unit 110 may add a special token. Among them, a class token may be added. The class token may be a token representing the overall characteristics of the amino acid sequence that is the input sentence.

The position information encoding unit 120 may embed position information of each token of the tokenized amino acid sequence in the tokenization unit 110 into an initial embedding vector, that is, the tokenized amino acid sequence and then may input the embedded position information into the artificial intelligence unit 130.

The artificial intelligence unit 130 may use a natural language processing (NLP) model.

The natural language processing model may be learned by a large amount of unlabeled text to process and analyze human language. The natural language processing model may refer to a first learned artificial intelligence model.

The NLP model may learn individual words so that related words are located close together in a linguistically meaningful embedding space through unsupervised learning.

This word embedding may help infer the meaning of words with multiple definitions depending on the context.

Examples of such NLP models may include transformers and bidirectional encoder representation from transformers (BERTs).

After learning the complex grammar and semantics of a language through unsupervised learning, additional supervised learning may be performed with labeled data to perform language-related downstream tasks such as machine translation, sentiment analysis, and question-answering. This is called fine tuning. The artificial intelligence model learned using the labeled data may refer to a second learned artificial intelligence model.

The artificial intelligence unit 130 may be similar to the unsupervised learning and the downstream tasks. For example, after learning the grammar and characteristics of the amino acid sequence that builds a valid protein by treating the protein sequence as the language and using the NLP model, the protein-related downstream tasks may be performed.

First, the artificial intelligence unit 130 may perform the unsupervised learning unrelated to the antimicrobial peptide function by using large amounts of protein sequences.

The fine-tuning may be performed next using labeled AMP and non-AMP amino acid sequences.

In the present invention, the fine tuning may be performed with Veltri et al.'s 1776 AMPs and 1776 non-AMP s (Veltri D, Kamath U, Shehu A. Deep learning improves antimicrobial peptide recognition. Bioinformatics. 2018; 34:2740-7.), but is not limited thereto.

The artificial intelligence unit 130 may be configured as a transformer or BERT model capable of implementing the NLP model.

The BERT model of the artificial intelligence unit 130 may utilize a self-attention mechanism in which an encoder module calculates how related each amino acid is to other amino acids in the given amino acid sequence by applying the concept of query, key, and value. All the query, key, and value vectors may have a weight matrix that is updated through learning.

If a single attention value vector is used when using the self-attention, a limitation in which the vector does not accurately represent a relationship between the amino acids in the amino acid sequence may occur. For example, meaningless results in which a specific amino acid's relationship with itself is evaluated too highly may occur.

To prevent this limitation, the artificial intelligence unit 130 according to an embodiment may apply the multi-head attention concept.

The attention vector may be calculated as the following equation.

$$Attention ( Q , K , V ) = softmax ( \frac{{QK}^{T}}{\sqrt{{d}_{k}}} ) V$$

Then, the multi-head attention may be calculated as the following equation.

MultiHead(Q,K,V)=Concat(Attention₁, . . . ,Attentionₙ)W⁰

As described above, the multi-head attention may generate a final embedding vector by calculating a weighted average of several attention vectors for each amino acid. The final embedding vector may contain useful information for every token.

The output unit 140 may finally determine the antimicrobial peptide function based on the final embedding vector.

The output unit 140 may be provided as a dense layer, that is, a fully connected (FC) layer to determine the antimicrobial peptide function of the amino acid sequence.

For this, the output unit 140 may be learned using a class token vector among the final sequence embedding vectors generated by passing the tokenized amino acid sequence through the artificial intelligence unit 130.

The trained output unit 140 may determine whether antimicrobial peptide function is present or not by calculating the class token vector among the final sequence embedding vectors of the amino acid sequence using the FC layer.

To measure the performance of the apparatus 100 for predicting the antimicrobial peptide function according to an embodiment, a data set consisting of antimicrobial peptide sequences (AMP sequence, source: Kang X, Dong F, Shi C, Liu S, Sun J, Chen J, et al. DRAMP 2.0, an updated data repository of antimicrobial peptides. Scientific Data. 2019; 6:1-10.) and non-antimicrobial peptide sequences (Non-AMP sequence, source: Bhadra P, Yan J, Li J, Fong S, Siu SW. AmPEP: sequence-based prediction of antimicrobial peptides using distribution patterns of amino acid properties and random forest. Sci Rep. 2018; 8: 1-10.) was used.

The function prediction performance of the model (AMP-BERT) according to an embodiment and the models according to the related art, which are measured using the data set, is shown in Table 1 below.

TABLE 1

| Model | SN | SP | F1 | ACC | AUROC | AUPR |
|---|---|---|---|---|---|---|
| CTD + RF | 0.6536 | 0.598 | 0.6495 | 0.63 | 0.6735 | 0.6586 |
| AMPScanner | 0.8412 | 0.6347 | 0.7722 | 0.742 | 0.7884 | 0.7621 |
| AMP-BERT | 0.8760 | 0.6352 | 0.7917 | 0.7604 | 0.8183 | 0.7866 |

It is seen that when the model (AMP-BERT) according to an embodiment is used, it shows the highest score in all indicators.

In addition, the performance evaluation table using only the amino acid sequences position-specific scoring matrix (PSSM) information is shown in Table 2 below.

Since the ACEP model that is one of the prior technologies essentially requires the PSSM information as the input, results of performance measured only using a partial subset of external data sets with the PSSM information for fair performance comparison is shown.

TABLE 2

| Model | SN | SP | F1 | ACC | AUROC | AUPR |
|---|---|---|---|---|---|---|
| ACEP | 0.874 | 0.6107 | 0.767 | 0.7398 | 0.8101 | 0.7411 |
| AMP-BERTsub | 0.8941 | 0.6330 | 0.8049 | 0.7610 | 0.8445 | 0.7984 |

The results of predicting only the sequences with the PSSM information may also confirm that the present disclosure using the AMP-BERT model shows excellent performance.

As described above, the AMP-BERT model according to an embodiment may use the self-attention technology to find trends in amino acid sequences associated with the antimicrobial function of amino acids constituting antimicrobial peptides.

When the attention value output from the AMP-BERT model according to an embodiment is analyzed, it may be seen that the amino acids that actually cause the AMP function are emphasized for prediction.

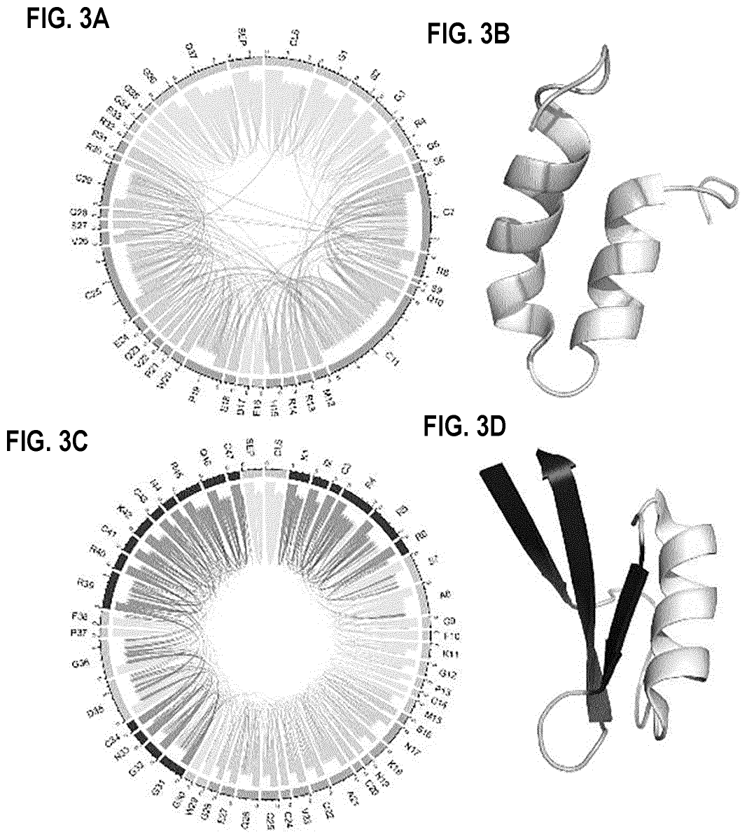

FIGS. 3A, 3B, 3C and 3D illustrate an example of attention value analysis results according to an embodiment.

Light green or blue amino acid portions having high attention values in FIG. 3A may represent a hydrophobic core and an alpha helix, which are important structures in AMP function shown in FIG. 3B, and blue and navy blue portions having high attention values in FIG. 3C may represent an alpha helix and a beta sheet.

Therefore, the apparatus for predicting antimicrobial peptide function according to an embodiment may predict antimicrobial peptide function with higher performance than the existing prediction model by using artificial intelligence, especially the natural language processing models and also analyze and visually confirm which amino acids contribute to antimicrobial peptide function through the attention mechanism.

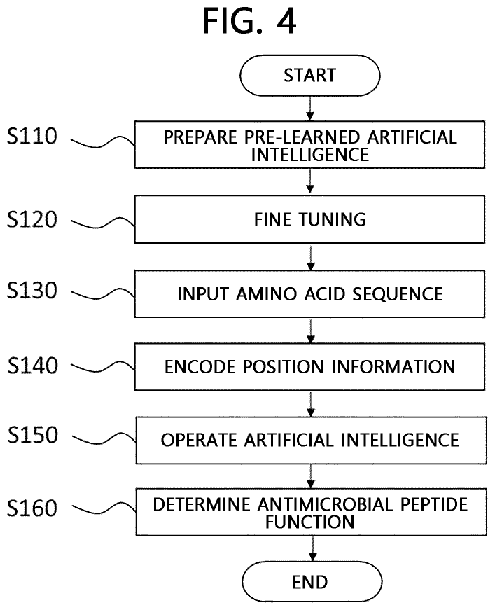

FIG. 4 is a schematic flowchart illustrating a method for predicting antimicrobial peptide function using artificial intelligence according to another embodiment.

A method for predicting antimicrobial peptide function according to an embodiment may be performed by a controller including one or more processors and a memory.

First, the artificial intelligence of the controller is prepared as a natural language processing (NLP) model that is pre-trained by large-scale unlabeled protein sequences (S110).

Examples of such NLP models may include transformers and bidirectional encoder representation from transformers (BERTs).

The artificial intelligence of the controller may learn grammars and characteristics of an amino acid sequence that builds a valid protein using the NLP model and then be further trained to perform protein-related downstream tasks (S120).

This additional learning process is referred to as fine tuning.

The fine-tuning, i.e., the additional learning may be implemented using labeled AMP and non-AMP amino acid sequences.

The amino acid sequence for determining the AMP function is input to the controller for which the pre-training and fine tuning have been completed (S130).

The amino acid sequence is tokenized as an input of a natural language processing model, and just as the natural language processing model uses each word in a sentence as a token, the artificial intelligence according to an embodiment may treat each amino acid in the input amino acid sequence as a word.

In addition, in addition to each amino acid in the amino acid sequence, a special token such as a class token representing the characteristics of the entire amino acid sequence are added to complete tokenization.

The position information of each token of the tokenized amino acid sequence is embedded in an initial embedding vector, that is, the tokenized amino acid sequence (S140).

The artificial intelligence of the controller, which has completed pre-training and fine tuning, performs a vector operation on the tokenized amino acid sequence to generate final sequence embedding (S150).

Here, the artificial intelligence model utilizes a self-attention mechanism that calculates how related each amino acid in the tokenized amino acid sequence is to other amino acids.

In addition, since the self-attention may produce meaningless results in which a specific amino acid is evaluated in its relationship with itself too highly, the multi-head attention concept may be applied to obtain a final embedding vector by calculating a weighted average of multiple attention vectors for each amino acid.

As a result, antimicrobial peptide function is predicted using vector information of the class token (or class token vector information) of the amino acid sequence embedding vectors finally generated through artificial intelligence (S160).

From the embedding vectors of all tokens, only the class token vector information is passed through a dense layer, that is, a fully connected (FC) layer, and antimicrobial peptide function is determined by binary classification.

As described above, in the apparatus and method for predicting antimicrobial peptide function using the artificial intelligence according to an embodiment, the large-scale protein sequences may be pre-trained using the natural language processing model, and the artificial intelligence may be additionally trained by the antimicrobial and non-antimicrobial peptide sequences to improve the performance of determining antimicrobial peptide function.

According to the present disclosure, it may have the effect of further increasing the accuracy of determining antimicrobial peptide function by pre-training the artificial intelligence, which is a bidirectional encoder representation from transformer (BERT)-based model, through unsupervised learning using large amounts of protein sequences and additionally training (fine turning) using labeled antimicrobial and non-antimicrobial peptide sequences.

In addition, there may be the advantage in saving the time and cost of antimicrobial screening by predicting antimicrobial peptide function based on the computer rather than the laboratory.

Although effects are not considered herein, the effects described in this specification and their provisional effects, which are expected by the technical features of the present invention, may be considered as the effects described in this specification.

The scope of the present invention is not limited to the description and expression of the embodiments explicitly described above. Further, it will be understood that the protective scope of the present invention is not limited by obvious modifications or substitutions in the technical fields of the present invention.

What is claimed is:

1. An apparatus for predicting antimicrobial peptide function using artificial intelligence including a controller, which comprises one or more processors and a memory,
wherein the controller comprises:
a tokenization unit configured to tokenize an input amino acid sequence for each amino acid and add a class token representing overall characteristics of the amino acid sequence to the tokenized amino acid sequence;
an artificial intelligence unit configured to perform a vector operation on the tokenized amino acid sequence for each token so as to generate a final sequence embedding vector by being pre-trained by a natural language processing (NLP) model by inputting unlabeled protein sequences and additionally trained by labeled antimicrobial peptide (AMP) and non-antimicrobial peptides (non-AMP); and
an output unit configured to determine the antimicrobial peptide function of the input amino acid sequence by performing a fully connected (FC) layer operation on the sequence embedding vector for the class token of the final sequence embedding vector.

2. The apparatus according to claim 1, wherein the artificial intelligence unit comprises a bidirectional encoder representation from transformer (BERT) model.

3. The apparatus according to claim 1, further comprising a position information encoding unit configured to encode position information of each token in the sequence tokenized by the tokenization unit.

4. The apparatus according to claim 1, wherein the artificial intelligence unit is configured to calculate how related each token of the tokenized sequence is to other tokens by a self-attention mechanism.

5. The apparatus according to claim 1, wherein the artificial intelligence unit is configured to generate the final sequence embedding vector by a multi-head attention mechanism that applies a weighted average of a plurality of attention vectors to each token of the tokenized sequence.

6. A method for predicting antimicrobial peptide function using artificial intelligence, which is performed by a controller comprising one or more processors and a memory, the method comprising:
preparing the artificial intelligence of the controller, which is a pre-trained natural language processing model, using large-scale unlabeled protein sequences as input;
additionally training the artificial intelligence using labeled antimicrobial peptide (AMP) sequences and non-antimicrobial peptide (non-AMP) sequences;
tokenizing the input amino acid sequence for each amino acid and adding a class token that represents overall characteristics of the amino acid sequence to the tokenized amino acid sequence to input the class token;
generating a final sequence embedding vector by performing a vector operation on the tokenized amino acid sequence, to which the class token is added for each token, by the artificial intelligence; and
determining the antimicrobial peptide function of the input amino acid sequence by performing a fully connected (FC) layer operation on the sequence embedding vector for the class token of the final sequence embedding vector.

7. The method according to claim 6, wherein the artificial intelligence of the controller comprises a bidirectional encoder representation from transformer (BERT) model.

8. The method according to claim 6, further comprising, after the inputting of the class token, encoding the positional information of each token in the tokenized amino acid sequence.

9. The method according to claim 6, wherein the artificial intelligence of the controller calculates how related each token of the tokenized sequence is to other tokens by a self-attention mechanism.

10. The method according to claim 6, wherein the artificial intelligence of the controller generates the final sequence embedding vector by a multi-head attention mechanism that applies a weighted average of a plurality of attention vectors to each token of the tokenized sequence.

11. A method for predicting antimicrobial peptide function using artificial intelligence, the method comprising:
preparing a first learned artificial intelligence model by inputting a plurality of unlabeled protein sequences;
performing second learning on the first learned artificial intelligence model to provide a second learned artificial intelligence model by using labeled antimicrobial peptide (AMP) sequences and non-antimicrobial peptide (non-AMP) sequences; and
inputting the amino acid sequence into the second learned artificial intelligence model to determine the antimicrobial peptide function of the input amino acid sequence.

12. The method according to claim 11, wherein the first learned artificial intelligence model uses artificial intelligence that is a pre-trained natural language processing model.

13. The method according to claim 11, further comprising:
tokenizing the input amino acid sequence for each amino acid and adding a class token that represents overall characteristics of the amino acid sequence to the tokenized amino acid sequence to input the amino acid sequence into the second learned artificial intelligence model;
generating a final sequence embedding vector by performing a vector operation on the tokenized amino acid sequence, to which the class token has been added for each token, by the second learned artificial intelligence model; and
determining the antimicrobial peptide function of the input amino acid sequence by performing a fully connected (FC) layer operation on the sequence embedding vector for the class token of the final sequence embedding vector.

14. The method according to claim 13, further comprising, after the inputting of the class token, encoding the positional information of each token in the tokenized amino acid sequence.

15. The method according to claim 13, wherein in the generating of the final sequence embedding vector, the second learned artificial intelligence model calculates how related each token of the tokenized sequence is to other tokens by a self-attention mechanism.

16. The method according to claim 15, wherein the self-attention mechanism calculates a degree of relationship between each amino acid and other amino acids in the input amino acid sequence by using queries, keys, and values.

17. The method according to claim 15, wherein, in the generating of the final sequence embedding vector, the second learned artificial intelligence model generates the final sequence embedding vector by a multi-head attention mechanism that applies a weighted average of a plurality of attention vectors to each token of the tokenized sequence.

18. The method according to claim 17, wherein the attention vector of the multi-head attention mechanism is expressed as following equation:
$$Attention ( Q , K , V ) = softmax ( \frac{{QK}^{T}}{\sqrt{{d}_{k}}} ) V$$
where, Q is the query, K is the key, and V is the value.

19. The method according to claim 17, wherein the multi-head attention of the multi-head attention mechanism calculates a weighted average of several attention vectors for each amino acid to generate the final embedding vector.

20. The method according to claim 18, wherein the multi-head attention of the multi-head attention mechanism is expressed as following equation:
Multihead(Q,K,V)=Concat(Attention₁, . . . ,Attentionₙ)W⁰.
